(19) United States
(12) Patent Application Publication
(10) Pub. No.: US 2017/0153322 A1
(43) Pub. Date: Jun. 1, 2017

(54) ULTRASONIC IMAGING DEVICE AND METHOD OF GENERATING ULTRASONIC IMAGE

(71) Applicant: SEIKO EPSON CORPORATION, Tokyo (JP)

(72) Inventor: Masaki HAYASHI, Matsumoto-shi (JP)

(73) Assignee: SEIKO EPSON CORPORATION, Tokyo (JP)

(21) Appl. No.: 15/360,828

(22) Filed: Nov. 23, 2016

(30) Foreign Application Priority Data
Nov. 26, 2015 (JP) 2015-230321

(51) Int. Cl. G01S 7/52; A61B 8/14; A61B 8/08; A61B 8/00; G01S 15/02; G01S 15/89

(52) U.S. Cl. CPC G01S 7/52047; G01S 15/02; G01S 7/5205; G01S 15/89; A61B 8/5269; A61B 8/54; A61B 8/14; A61B 8/5207

(57) ABSTRACT

An ultrasonic measurement control unit of a processing unit of an ultrasonic imaging device forms an ultrasonic measurement unit, which performs ultrasonic measurement, together with an ultrasonic wave transmitting and receiving unit. A desired sensitivity attenuation position setting unit sets a desired sensitivity attenuation position in a generated ultrasonic image. An image generation unit generates an ultrasonic image by performing a beam forming process in which received signals of elements input from the ultrasonic measurement control unit are added. In the beam forming process, a beam forming processing unit performs attenuation control to attenuate the signal strength of a received ultrasonic signal corresponding to the desired sensitivity attenuation position.

### BACKGROUND

1. Technical Field

The present invention relates to an ultrasonic imaging device generating an ultrasonic image.

2. Related Art

The related art discloses an ultrasonic imaging device that scans with ultrasonic beams via a probe in which multiple ultrasonic elements (oscillators) are arrayed, and images the pattern of the inside of a living body. In an imaging process, ultrasonic waves are transmitted from a body surface of a subject toward the inside of a living body, and a beam forming process, in which received signals (received ultrasonic signals) of the elements for reflected waves are phase added, is performed.

An artifact (virtual image) which is a problems in generating an ultrasonic image is caused by a side lobe. If a strong reflecting object is present in the direction of the side lobe, reflected waves from the reflecting object are received as undesired waves, thereby causing deterioration in image quality. JP-A-2015-71028 discloses a beam forming process, by which direction constraint is applied and an ultrasonic image does not have sensitivity to undesired waves from directions other than a desired direction, as technology for solving such a problem.

According to the technology disclosed in JP-A-2015-71028, a weight used to add received signals is determined such that the sensitivities of the undesired waves from the direction other than the desired direction are minimized, and a normal process is performed in the directions other than the desired direction. For this reason, in a case where an artifact occurs due to a side lobe, a process to focus on the artifact part is not performed.

### SUMMARY

An advantage of some aspects of the invention is that an artifact caused by a side lobe is effectively reduced, and deterioration of the quality of an ultrasonic image is prevented.

A first aspect of the invention is directed to an ultrasonic imaging device that generates an ultrasonic image by performing a beam forming process in which received ultrasonic signals are added, the device including: a position setting unit that sets a desired sensitivity attenuation position in a generated ultrasonic image; and a computational processing unit that executes the beam forming process while performing attenuation control to attenuate the signal strength of a received ultrasonic signal corresponding to the desired sensitivity attenuation position.

Another aspect of the invention is directed to a method of generating an ultrasonic image in which an ultrasonic image is generated by performing a beam forming process in which received ultrasonic signals are added, the method including: setting a desired sensitivity attenuation position in a generated ultrasonic image; and executing the beam forming process while performing attenuation control to attenuate the signal strength of a received ultrasonic signal corresponding to the desired sensitivity attenuation position.

According to the first aspect and the like, in the beam forming process in which received ultrasonic signals are added, it is possible to perform attenuation control to attenuate the signal strength of a received ultrasonic signal corresponding to a desired sensitivity attenuation position. Accordingly, it is possible to perform the beam forming process while attenuating the signal strength of a received ultrasonic signal arrived from a position inside a living body which is represented by the desired sensitivity attenuation position. As a result, it is possible to effectively reduce an artifact caused by a side lobe, and to prevent deterioration of the quality of an ultrasonic image.

As a second aspect, the ultrasonic imaging device of the first aspect may be configured such that the beam forming process is performed for each scanning line of ultrasonic waves, and the computational processing unit specifies a corresponding spot of the desired sensitivity attenuation position on a processing target scanning line, and performs the attenuation control when the beam forming process is performed for the corresponding spot.

According to the second aspect, it is possible to specify a corresponding spot of a desired sensitivity attenuation position on a processing target scanning line. When a beam forming process is performed for the specified corresponding spot, it is possible to perform attenuation control to attenuate the signal strength of a received ultrasonic signal corresponding to the desired sensitivity attenuation position.

As a third aspect, the ultrasonic imaging device of the second aspect may be configured such that the computational processing unit detects a distance between a base point of the processing target scanning line and the desired sensitivity attenuation position, and specifies the corresponding spot on the processing target scanning line based on the distance.

According to the third aspect, it is possible to specify a corresponding spot of a desired sensitivity attenuation position on a processing target scanning line based on the distance from a base point of a processing target scanning line to the desired sensitivity attenuation position.

As a fourth aspect, the ultrasonic imaging device of the second or third aspect may be configured such that the computational processing unit detects the direction of the desired sensitivity attenuation position with respect to the processing target scanning line, calculates an addition weight such that a null point of the receive sensitivity of the received ultrasonic signal faces the direction of the desired sensitivity attenuation position, and adds received ultrasonic signals in a weighted manner using the calculated addition weight.

According to the fourth aspect, when a beam forming process is performed for a corresponding spot of a desired sensitivity attenuation position on a processing target scanning line, it is possible to calculate an addition weight such that a null point of the receive strength of a received ultrasonic signal faces the direction of the desired sensitivity attenuation position with respect to the processing target scanning line. It is possible to add received ultrasonic signals in a weighted manner using the calculated addition weight.

As a fifth aspect, the ultrasonic imaging device of the first to fourth aspects may be configured such that the position setting unit sets multiple desired sensitivity attenuation positions in a predetermined region inside the generated ultrasonic image.

According to the fifth aspect, it is possible to set multiple desired sensitivity attenuation positions in a predetermined region inside a generated ultrasonic image.

As a sixth aspect, the ultrasonic imaging device of the first to fifth aspects may be configured such that the ultrasonic imaging device further includes a provisional image generation unit that generates a provisional ultrasonic image based on the received ultrasonic signals, and the position setting unit extracts a strong reflecting region from the provisional ultrasonic image, and sets the desired sensitivity attenuation position in the strong reflecting region.

According to the sixth aspect, it is possible to generate a provisional ultrasonic image, and to set a desired sensitivity attenuation position in a strong reflecting region inside the generated provisional ultrasonic image. Accordingly, it is possible to perform a beam forming process while attenuating the signal strength of received ultrasonic signals reflected by a strong reflecting object in a living body.

As a seventh aspect, the ultrasonic imaging device of the first to sixth aspects may be configured such that the ultrasonic imaging device further includes a provisional image generation unit that generates a provisional ultrasonic image based on the received ultrasonic signals, and the position setting unit receives an operation input indicating a position in the provisional ultrasonic image, and sets the desired sensitivity attenuation position.

According to the seventh aspect, it is possible to generate a provisional ultrasonic image, and to set a position in the generated provisional ultrasonic image, which is indicated by an operation input, as a desired sensitivity attenuation position.

### BRIEF DESCRIPTION OF THE DRAWINGS

The invention will be described with reference to the accompanying drawings, wherein like numbers reference like elements.

FIG. 1 is a view illustrating an example of a system configuration of an ultrasonic imaging device.

FIG. 2 is a view illustrating the principle of a beam forming process of an embodiment.

FIG. 3 is a graph illustrating the sensitivities of ultrasonic beams.

FIG. 4 is a block diagram illustrating an example of a functional configuration of an ultrasonic imaging device.

FIG. 5 is a flowchart illustrating the flow of a process of generating an ultrasonic image.

FIG. 6 is a view illustrating a setting example in which multiple desired sensitivity attenuation positions are set.

FIG. 7 is a view illustrating another setting example in which multiple desired sensitivity attenuation positions are set.

### DESCRIPTION OF EXEMPLARY EMBODIMENTS

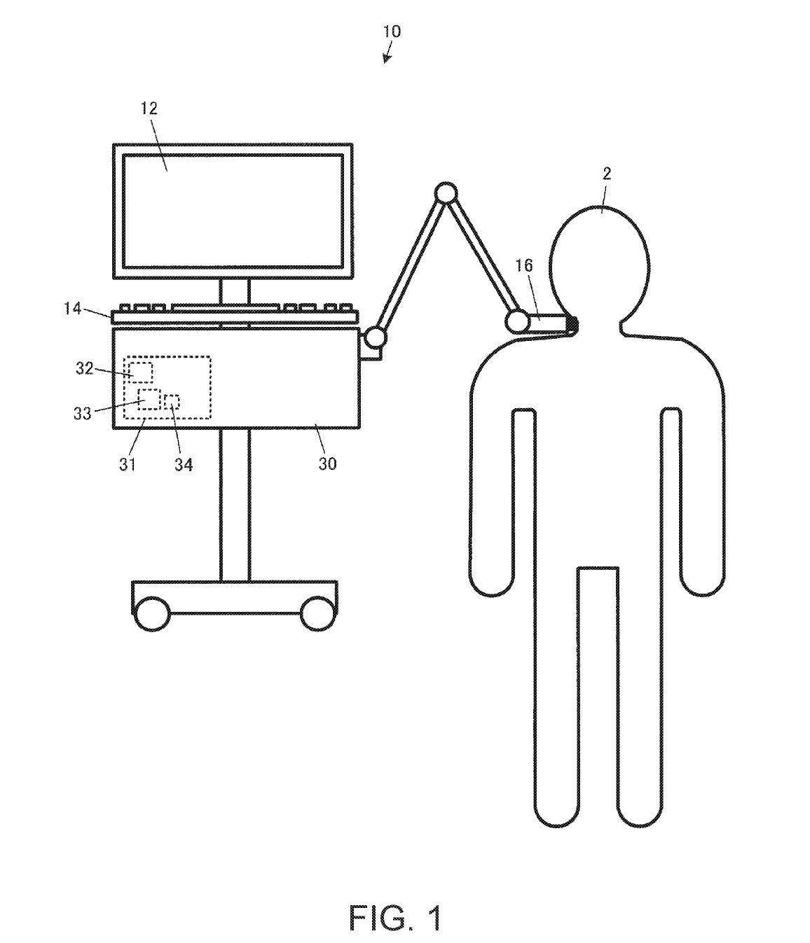

FIG. 1 is a view illustrating an example of a system configuration of an ultrasonic imaging device 10 of an embodiment. The ultrasonic imaging device 10 is a device that acquires biological information regarding a subject 2 using ultrasonic waves. The ultrasonic imaging device 10 includes a touch panel 12 that serves as both a unit for displaying measurement results or operation information as images and a unit through which operations are input; a keyboard 14 through which operations are input; an ultrasonic probe 16; and a processing device 30.

A control substrate 31 is mounted in the processing device 30. The processing device 30 is connected to parts of the device such as the touch panel 12, the keyboard 14, and the ultrasonic probe 16 in such a way as to be capable of transmitting to and receiving signals therefrom. The following components are mounted on the control substrate 31: various integrated circuits such as a central processing unit (CPU) 32, an application specific integrated circuit (ASIC), and a field programmable gate array (FPGA); a storage medium 33 such as an IC memory or a hard disk; and a communication IC 34 that realizes data communication with external devices. The processing device 30 performs processes including ultrasonic measurement required to acquire biological information by causing the CPU 32 to execute a program stored in the storage medium 33.

Specifically, the ultrasonic imaging device 10 performs ultrasonic measurement by transmitting ultrasonic beams to the subject 2 from the ultrasonic probe 16, and receiving reflected waves via control performed by the processing device 30. Reflected wave data such as positional information regarding or a change over time in an in vivo structure of the subject 2 by amplifying and processing received signals of the reflected waves. The ultrasonic measurement is repeatedly performed at predetermined intervals. A measurement unit is referred to as a “frame”.

The reflected wave data contains images in modes such as so-called A mode, B mode, M mode, and color Doppler mode. The A mode is a mode of displaying the amplitudes (A mode image) of reflected waves in which a row of sampling points for received signals along a scanning line direction of an ultrasonic beam is represented on a first axis and the received signal strength of a reflected wave at each sampling point is represented on a second axis. The B mode is a mode of displaying a two-dimensional ultrasonic image (B mode image) of an in vivo structure which is visualized by converting the amplitudes (A mode image) of reflected waves, which are obtained by scanning with ultrasonic beams a predetermined probe scanning range, into luminance values.

### Principle

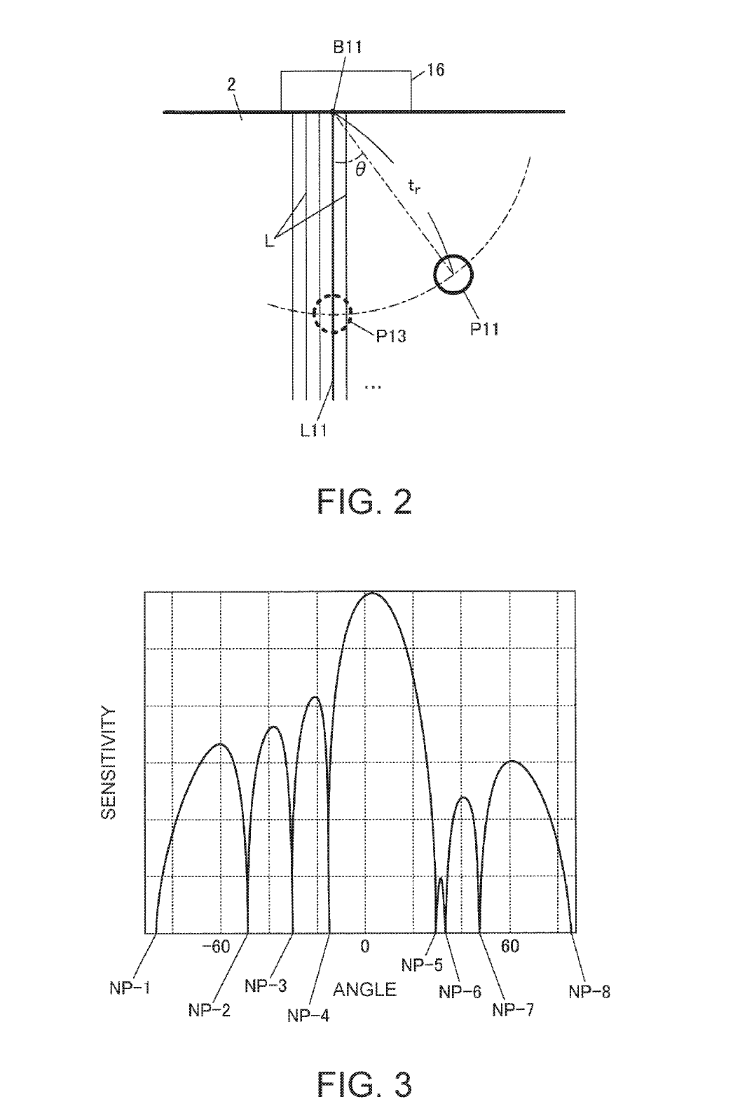

FIG. 2 is a view illustrating the principle of abeam forming process which is performed to generate an ultrasonic image in the embodiment. FIG. 2 schematically illustrates a state in which the ultrasonic probe 16 is brought into contact with a body surface of the subject 2, and ultrasonic measurement is performed. Multiple ultrasonic elements (not illustrated) are built into the ultrasonic probe 16. The ultrasonic measurement is performed via a so-called linear scanning method in which ultrasonic beams are transmitted and received along multiple parallel scanning lines L while shifting the position of incidence of an ultrasonic beam in a longitudinal direction of the ultrasonic probe 16 in which the ultrasonic elements are arrayed.

A main emitted ultrasonic beam represents a main lobe that is emitted along the scanning line L and has a high sound pressure. Due to characteristics of ultrasonic waves, in addition to the main lobe, an ultrasonic beam (side lobe) having a low sensitivity is generated in a direction deviating obliquely from the direction of the scanning line L. If a strong reflecting object is present in the direction of the side lobe, there is a problem in that a virtual image of the strong reflecting object appears in an ultrasonic image. An example of the strong reflecting object is a bone.

Multiple null points are present in the sensitivities of ultrasonic beams (more specifically, the sensitivities of received reflected waves of ultrasonic waves, but, for the sake of easy understanding, will be described as the sensitivities of ultrasonic beams) in the directions deviating from the scanning lines L. FIG. 3 is a graph illustrating the sensitivity of an ultrasonic beam in each direction with the direction of the scanning line L set to 0 degrees. Points NP (NP-1 to NP-8) where the sensitivity illustrated in FIG. 3 falls rapidly and becomes the minimum represent null points. Accordingly, if the null point NP faces the direction of the strong reflecting object, that is, a side lobe is not emitted in the direction of the strong reflecting object, it is possible to prevent the reception of reflected waves from the strong object.

In the embodiment, after ultrasonic measurement is performed, first, the position of a strong reflecting object present inside a living body, that is, the position of the strong reflecting object in a generated ultrasonic image is set as a desired sensitivity attenuation position. According to a procedure, for example, a typical image generation process is executed based on the result of the ultrasonic measurement, and a provisional ultrasonic image is generated. Subsequently, a strong reflecting region is extracted from the generated provisional ultrasonic image. The extraction of the strong reflecting region can be performed via a well-known technique. A high luminance region may be extracted from the provisional ultrasonic image, and set as a strong reflecting region. Alternatively, a strong reflecting region may be extracted based on the signal strength of received signals obtained in the ultrasonic measurement. A point in the strong reflecting region is set as a desired sensitivity attenuation position. The method of determining the desired sensitivity attenuation position is not limited to a specific method. One point may be randomly selected from the strong reflecting region, or a central position in the strong reflecting region may be set as the desired sensitivity attenuation position.

If the desired sensitivity attenuation position has been set, attenuation control for attenuating the signal strength of a received signal corresponding to the desired sensitivity attenuation position is performed, and a beam forming process is executed. Specifically, the beam forming process has all scanning lines as sequential processing targets, and is a process of adding received signals of the elements for each sampling point on a scanning line which is a processing target (hereinafter, referred to as a “processing target line”). In the adding process, apart from a corresponding spot specified on the processing target line which is a spot corresponding to the desired sensitivity attenuation position, received signals of the elements for sampling points are added in a weighted manner using a predetermined fixed addition weight. In contrast, in order for the null point NP to face the direction of the desired sensitivity attenuation position (direction of the strong reflecting object inside the living body which is represented by the desired sensitivity attenuation position), the addition weight used in adding the received signals is dynamically changed, and addition is performed for a sampling point, which is the corresponding spot, in a weighted manner using the changed addition weight. So-called adaptive beam forming is performed.

The corresponding spot of the desired sensitivity attenuation position on the processing target line represents a sampling point on the processing target line which is affected by reflected waves from the strong reflecting object represented by the desired sensitivity attenuation position, that is, a sampling point among the sampling points on the processing target line at which an arrival time of a reflected wave is the same as that of a reflected wave from the desired sensitivity attenuation position. Specifically, the distance (inter-desired position distance) between a base point of the processing target line which is the position of incidence of an ultrasonic wave and the desired sensitivity attenuation position is calculated. A sampling point on the processing target line, the distance of which from the base point is the inter-desired position distance is specified as the corresponding spot.

For example, a position P11 illustrated by a solid circle in FIG. 2 is assumed to be a desired sensitivity attenuation position. If a scanning line L11 is assumed to be a processing target line, an inter-desired position tᵣ between a base point B11 of the processing target line L11 and the desired sensitivity attenuation position P11 is calculated. A sampling point on the processing target line L11, the distance of which from the base point B11 is the same distance as the inter-desired position distance tᵣ for the desired sensitivity attenuation position P11 is assumed to be a corresponding spot P13.

A beam forming process (adaptive beam forming) for a corresponding spot will be described in detail later. For this process, the direction (desired position azimuth) of the desired sensitivity attenuation position with respect to the processing target line is calculated along with the inter-desired position distance tᵣ. That is, as illustrated in FIG. 2, an angle θ between the processing target line L11 and the direction of the desired sensitivity attenuation position P11 is calculated as a desired position azimuth, and is used in a beam forming process for the corresponding spot P13.

### Functional Configuration

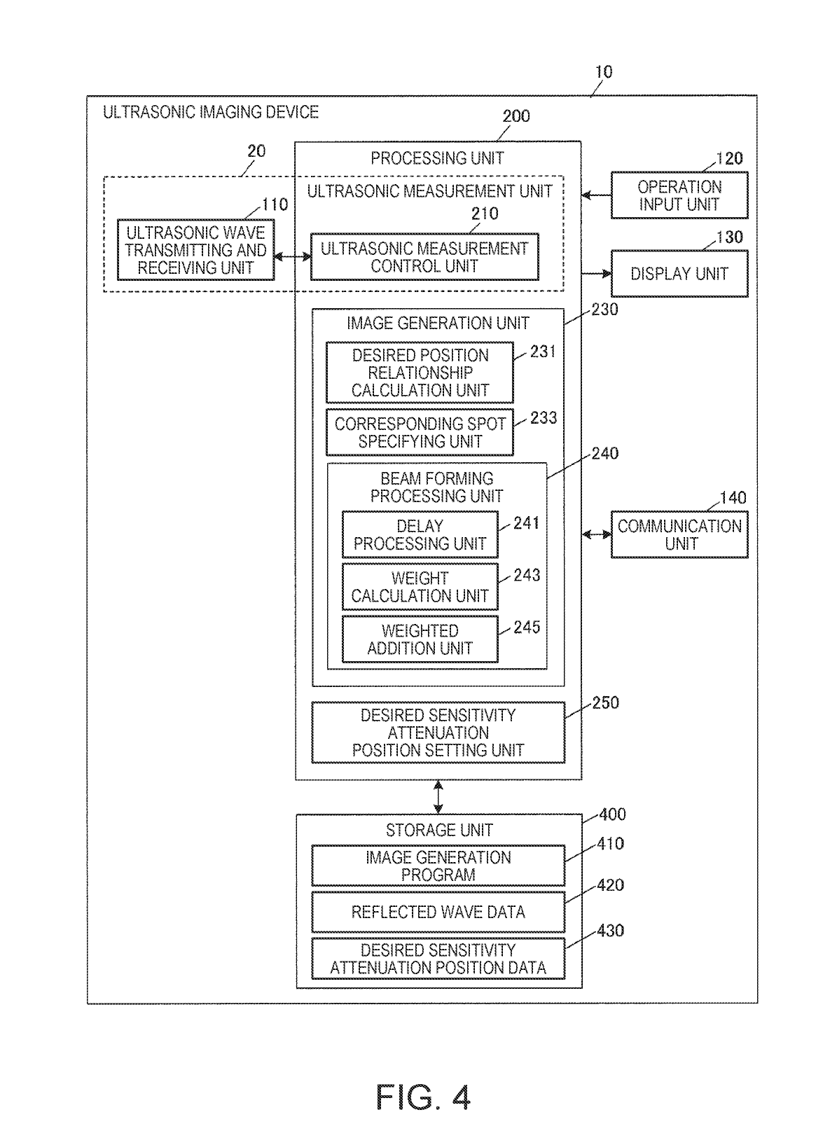

FIG. 4 is a block diagram illustrating an example of a functional configuration of the ultrasonic imaging device 10. The ultrasonic imaging device 10 includes an ultrasonic wave transmitting and receiving unit 110; an operation input unit 120; a display unit 130; a communication unit 140; a processing unit 200; and a storage unit 400.

The ultrasonic wave transmitting and receiving unit 110 transmits ultrasonic waves at a pulse voltage output from an ultrasonic measurement control unit 210 of the processing unit 200. The ultrasonic wave transmitting and receiving unit 110 receives reflected waves of the transmitted ultrasonic waves, and outputs received signals to the ultrasonic measurement control unit 210. The ultrasonic wave transmitting and receiving unit 110 includes multiple ultrasonic elements, and in FIG. 1, the ultrasonic probe 16 corresponds to the ultrasonic wave transmitting and receiving unit 110.

The operation input unit 120 receives various operation inputs from a user, and outputs an operation input signal, which corresponds to an operation input, to the processing unit 200. The operation input unit 120 can be realized by a button switch, a lever switch, a dial switch, a track pad, a mouse, or the like. In FIG. 1, the touch panel 12 and the keyboard 14 correspond to the operation input unit 120.

The display unit 130 is realized by a display device such as a liquid crystal display (LCD), and performs various displays based on a display signal from the processing unit 200. In FIG. 1, the touch panel 12 corresponds to the display unit 130.

The communication unit 140 is a communication device that transmits to and receives data from external devices under control performed by the processing unit 200. The communication unit 140 is capable of performing communication in various methods, for example, via wired connection using a cable conforming to predetermined communication standards, via connection using an intermediate device referred to as a cradle or the like which also serves as a charger, or via wireless connection using wireless communication. In FIG. 1, the communication IC 34 corresponds to the communication unit 140.

The processing unit 200 is realized by electronic components, for example, microprocessors such as a CPU and a graphics processing unit (GPU), an ASIC, a FPGA, and an IC memory. The processing unit 200 controls the input and output of data between functional units, and calculates biological information regarding the subject 2 by executing various computational processes based on a predetermined program, data, operation input signals from the operation input unit 120, a received signal of each element, and the like from the ultrasonic wave transmitting and receiving unit 110. In FIG. 1, the processing device 30 and the control substrate 31 correspond to the processing unit 200.

The processing unit 200 includes the ultrasonic measurement control unit 210; an image generation unit 230; and a desired sensitivity attenuation position setting unit 250.

The ultrasonic measurement control unit 210 forms the ultrasonic measurement unit 20 together with the ultrasonic wave transmitting and receiving unit 110. The ultrasonic measurement unit 20 performs ultrasonic measurement. The ultrasonic measurement control unit 210 controls a transmission timing of an ultrasonic pulse from the ultrasonic wave transmitting and receiving unit 110, and generates and outputs a pulse voltage to the ultrasonic wave transmitting and receiving unit 110 at the transmission timing. The ultrasonic measurement control unit 210 adjusts an output timing of a pulse voltage to each element by performing a transmission delay process. The ultrasonic measurement control unit 210 amplifies and filters the received signal of each element input from the ultrasonic wave transmitting and receiving unit 110, and outputs the processed received signal (measurement result) of each element to the image generation unit 230.

The image generation unit 230 generates an ultrasonic image based on the received signals of the elements input from the ultrasonic measurement control unit 210. The image generation unit 230 includes a desired position relationship calculation unit 231; a corresponding spot specifying unit 233; and a beam forming processing unit 240.

The desired position relationship calculation unit 231 calculates the inter-desired position distance tᵣ between a base point of a processing target line and a desired sensitivity attenuation position, and calculates a desired position azimuth θ of the desired sensitivity attenuation position with respect to the processing target line.

The corresponding spot specifying unit 233 specifies a corresponding spot of the desired sensitivity attenuation position on the processing target line based on the inter-desired position distance tᵣ.

The beam forming processing unit 240 includes a delay processing unit 241; a weight calculation unit 243; and a weighted addition unit 245. The beam forming processing unit 240 performs a beam forming process for each processing target line.

The delay processing unit 241 performs a delay process in which the received signal of each element is multiplied by a delay of a predetermined delay time Dₘ for the element. The received signal of each element after the delay process is output to the beam forming processing unit 240.

The weight calculation unit 243 calculates an addition weight used to perform a beam forming process for a corresponding spot, based on the desired position azimuth θ. The beam forming process for a corresponding spot is performed in such a way that the weighted addition unit 245 adds the received signals of the elements after the delay process in a weighted manner using the addition weight calculated by the weight calculation unit 243.

The calculation of an addition weight will be described. If an addition weight of each element is assumed to be Wₘ, an output z [n] of the weighted addition unit 245 is the result of multiplying a received signal xₘ [n−Dₘ [n]] of each element after the delay process, which is input from the delay processing unit 241, by the addition weight Wₘ of the element, and adding the resultants. The output z [n] is represented by Expression (1). m represents the number of elements (channels). In the embodiment, an addition weight is calculated per element. If a group of multiple elements form one channel, and transmit and receive ultrasonic waves, the ultrasonic measurement control unit 210 outputs a received signal for each channel. In this case, an addition weight for each channel is calculated, and a received signal of each channel is multiplied by the addition weight. n represents the total number of samplings, and xₘ [n] represents a received signal of an m^{th} element for a sampling point n.

$$z [ n ] = {\sum }_{m = 0}^{M - 1} {w}_{m} [ n ] {x}_{m} [ n - {D}_{m} [ n ] ] \qquad ( 1 )$$

If Expression (1) is represented by vectors, Expression (1) is converted into Expressions (2) and (3). H represents a complex conjugate transpose, and * represents a complex conjugate.

$$\begin{matrix}z [ n ] = {w [ n ]}^{H} X [ n ] & ( 2 ) \\ w [ n ] = [ \begin{matrix}{w}_{0}^{*} [ n ] \\ {w}_{1}^{*} [ n ] \\ ⋮ \\ {w}_{M - 1}^{*} [ n ]\end{matrix} ] & ( 3 )\end{matrix}$$

A correlation matrix R [n] is given by Expressions (4) and (5).

R[N]E[X[N]X[N]^{T}] (4)

E[|Z[N]|²]=W[N]^{H}R[N]W[N] (5)

An addition weight used to perform beam forming on a corresponding spot is a value that minimizes a variance of z [n] in Expressions (4) and (5), and is obtained by solving a constrained minimization problem represented by Expressions (6) and (7). If a Lagrange undetermined multiplier method is used, an addition weight of each element is represented by Expression (8).

$$\begin{matrix}{min}_{w [ m ]} {w [ n ]}^{H} R [ n ] w [ n ] & ( 6 ) \\ subject to {w [ n ]}^{H} a = H & ( 7 ) \\ w [ n ] = \frac{{R [ n ]}^{- 1} a}{{a}^{H} {R [ n ]}^{- 1} a} {H}^{*} & ( 8 )\end{matrix}$$

a presents a steering vector, and H represents a constrained response vector. In the embodiment, phasing is already performed, and thus, the direction of a desired wave (main lobe) is assumed to be 0 degrees. Since the null point NP is at the desired position azimuth θ, the direction of an undesired wave is set at θ, and constrained conditions represented by Expressions (9), (10), and (11) are applied.

$$\begin{matrix}a = [ \begin{matrix}{a}_{0} & {a}_{θ}\end{matrix} ] & ( 9 ) \\ {a}_{θ} = ( \begin{matrix}1 & {e}^{- j π si n ( θ )} & {e}^{- j2π si n ( θ )} & … & {e}^{- j ( M - 1 ) π si n ( θ )}\end{matrix} ) & ( 10 ) \\ H = [ \begin{matrix}1 \\ 0\end{matrix} ] & ( 11 )\end{matrix}$$

The description returns to FIG. 4. The weighted addition unit 245 adds the received signals of the elements for sampling points apart from corresponding spots in a weighted manner using the fixed addition weight, and adds the received signals of the elements for the corresponding spots in a weighted manner using the addition weight calculated by the weight calculation unit 243.

The desired sensitivity attenuation position setting unit 250 generates a provisional ultrasonic image, extracts a strong reflecting region, and sets a desired sensitivity attenuation position in the generated ultrasonic image by selecting one point in the strong reflecting region.

The storage unit 400 is realized by a storage medium such as an IC memory, a hard disk, or an optical disk. The storage unit 400 stores in advance a program that realizes various functions of the ultrasonic imaging device 10 by operating the ultrasonic imaging device 10, and data used in the execution of the program, or temporarily stores data whenever processed. In FIG. 1, the storage medium 33 mounted on the control substrate 31 corresponds to the storage unit 400. Connection between the processing unit 200 and the storage unit 400 is not limited to connection via an internal bus circuit inside the device, and may be realized by a communication line such as a local area network (LAN) or Internet. In this case, the storage unit 400 may be realized by an external storage device separate from the ultrasonic imaging device 10.

The storage unit 400 stores an image generation program 410, reflected wave data 420, desired sensitivity attenuation position data 430, and the like.

The processing unit 200 realizes the functions of the ultrasonic measurement control unit 210, the image generation unit 230, the desired sensitivity attenuation position setting unit 250, and the like by reading and executing the image generation program 410. If these functional units are realized by hardware such as electronic circuits, it is possible to omit a portion of programs for realizing the functions.

The reflected wave data 420 stores reflected wave data obtained via ultrasonic measurement repeated for each frame. The reflected wave data 420 contains data of a B mode image for each frame which is an ultrasonic image. The desired sensitivity attenuation position data 430 stores desired sensitivity attenuation positions set by the desired sensitivity attenuation position setting unit 250.

### Flow of Process

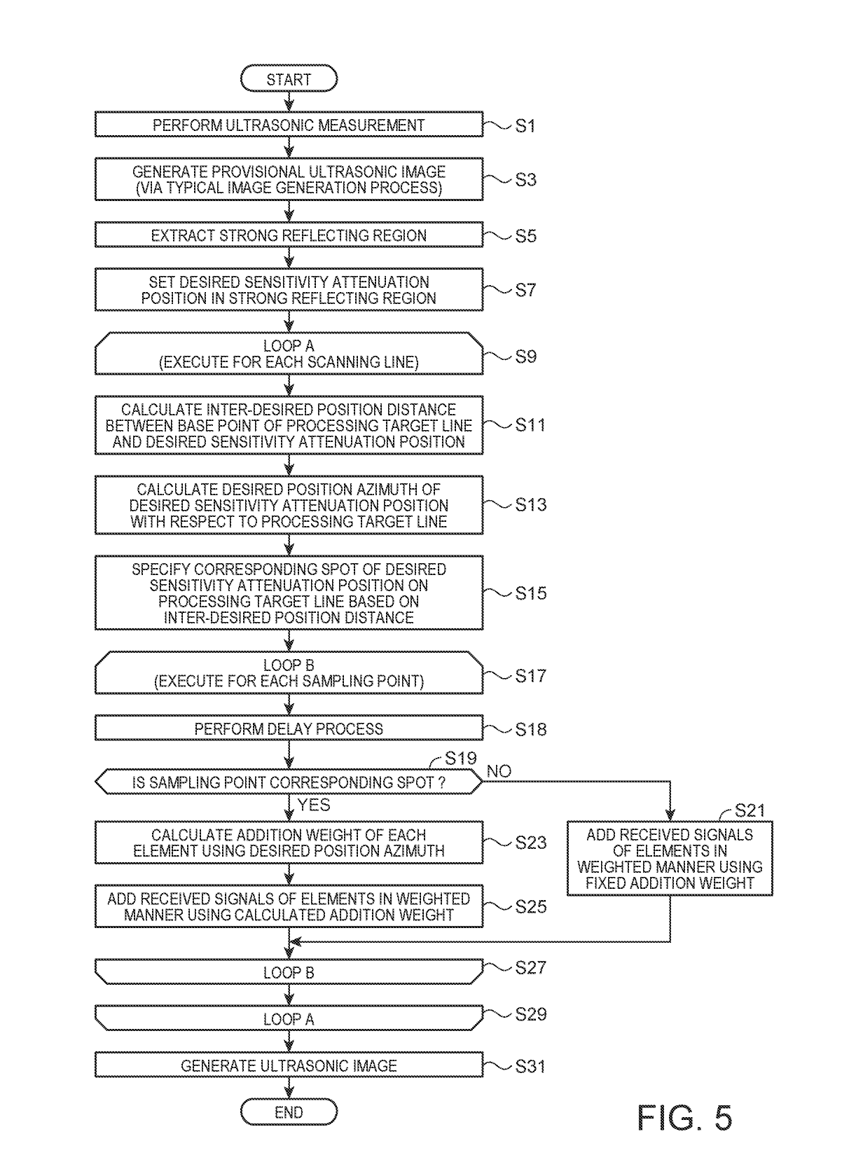

FIG. 5 is a flowchart illustrating the flow of a process of generating an ultrasonic image in the embodiment. If a user brings the ultrasonic probe 16 into contact with a body surface, and inputs a predetermined measurement start operation, the process described herein is started. The processing unit 200 operates each unit of the ultrasonic imaging device 10 by reading the image generation program 410 from the storage unit 400 and executing the image generation program 410. As a result, the process can be realized.

First, the ultrasonic measurement unit 20 performs ultrasonic measurement (Step S1). The image generation unit 230 generates a provisional ultrasonic image by performing a typical image generation process as a provisional image generation unit (Step S3). The typical image generation process is a process in which an ultrasonic image is generated via a technique in the related art. In abeam forming process, a fixed addition weight is used for all sampling points on each scanning line. Specifically, first, the delay processing unit 241 performs a delay process on a received signal of each element obtained from the ultrasonic measurement in Step S1. The weighted addition unit 245 uses the fixed addition weight as the addition weight Wₘ of each element, and adds the received signals of the elements after the delay process in a weighted manner according to Expression (1).

Subsequently, the desired sensitivity attenuation position setting unit 250 extracts a strong reflecting region from the provisional ultrasonic image (Step S5), and sets one desired sensitivity attenuation position in the strong reflecting region (Step S7).

If the desired sensitivity attenuation position is set, the beam forming process is performed by repeating the process of a loop A for each scanning line (Steps S9 to S29).

In the loop A, first, the desired position relationship calculation unit 231 calculates the inter-desired position distance tᵣ between a base point of a processing target line and a desired sensitivity attenuation position (Step S11), and calculates the desire position azimuth θ of the desired sensitivity attenuation position with respect to the processing target line (Step S13). The corresponding spot specifying unit 233 specifies a corresponding spot of the desired sensitivity attenuation position on the processing target line based on the inter-desired position distance tᵣ calculated in Step S11 (Step S15).

Subsequently, sampling is performed during a predetermined length of time for the processing target line using the results of the ultrasonic measurement performed in Step S1, and the process of a loop B is performed for each sampling point (Steps S17 to S27).

In the loop B, first, the delay processing unit 241 performs a delay process in which a received signal of each element is multiplied by a delay of the delay time Dₘ (Step S18). Subsequently, the process diverges according to whether a sampling point is the corresponding spot. If the sampling point is not the corresponding spot (Step S19: NO), the weighted addition unit 245 uses a fixed addition weight as the addition weight Wₘ of each element, and adds the received signals of the elements after the delay process in Step S18 in a weighted manner according to Expression (1) (Step S21). In contrast, if the sampling point is the corresponding spot (Step S19: YES), the weight calculation unit 243 calculates an addition weight of each element in the aforementioned manner using the desired position azimuth θ calculated in Step S13 (Step S23). The weighted addition unit 245 adds the received signals of the elements after the delay process in Step S18 in a weighted manner using the addition weight, which is calculated in Step S23, as the addition weight Wₘ of each element according to Expression (1) (Step S25).

If the process of the loop B is repeated, and the sampling for the processing target line ends, the process of the loop A for the processing target line ends. If the process of the loop A is performed for all the scanning lines which are processing targets, an ultrasonic image is generated by performing a necessary process on the obtained output z [n] of each sampling point (Step S31), and the process ends. The generated ultrasonic image is suitably controlled and displayed on the display unit 130.

As described above, according to the embodiment, it is possible to generate a provisional ultrasonic image in advance, to extract a strong reflecting region therefrom, and to set a desired sensitivity attenuation position in the strong reflecting region. When a beam forming process is performed for a corresponding spot of a desired sensitivity attenuation position on a processing target line, it is possible to calculate an addition weight for the null point NP to face the direction of the desired sensitivity attenuation position, and to add in a weighted manner a received signal of each element using the calculated addition weight. Accordingly, it is possible to perform the beam forming process while reliably attenuating the signal strength of a received signal arrived from a strong reflecting object inside a living body which is represented by the desired sensitivity attenuation position. As a result, it is possible to effectively reduce an artifact caused by a side lobe, and to prevent deterioration of the quality of an ultrasonic image.

The invention is not limited to the configuration of the embodiment in which a desired sensitivity attenuation position is automatically set in a strong reflecting region extracted from a provisional ultrasonic image. Alternatively, a desired sensitivity attenuation position may be manually set according to an operation input performed by a user. For example, the setting of a desired sensitivity attenuation position may be realized by performing control to display a provisional ultrasonic image, which is generated in Step S3 illustrated in FIG. 5, on the display unit 130, and receiving an operation of designating one point in the provisional ultrasonic image via the operation input unit 110. Alternatively, instead of a process of extracting a strong reflecting region, a user may manually designate the range of a strong reflecting region in a provisional ultrasonic image. A strong reflecting region may be extracted from a captured image of the same site obtained via a computed tomography (CT) scan, magnetic resonance imaging (MRI), or the like, and a desired sensitivity attenuation position may be set therein.

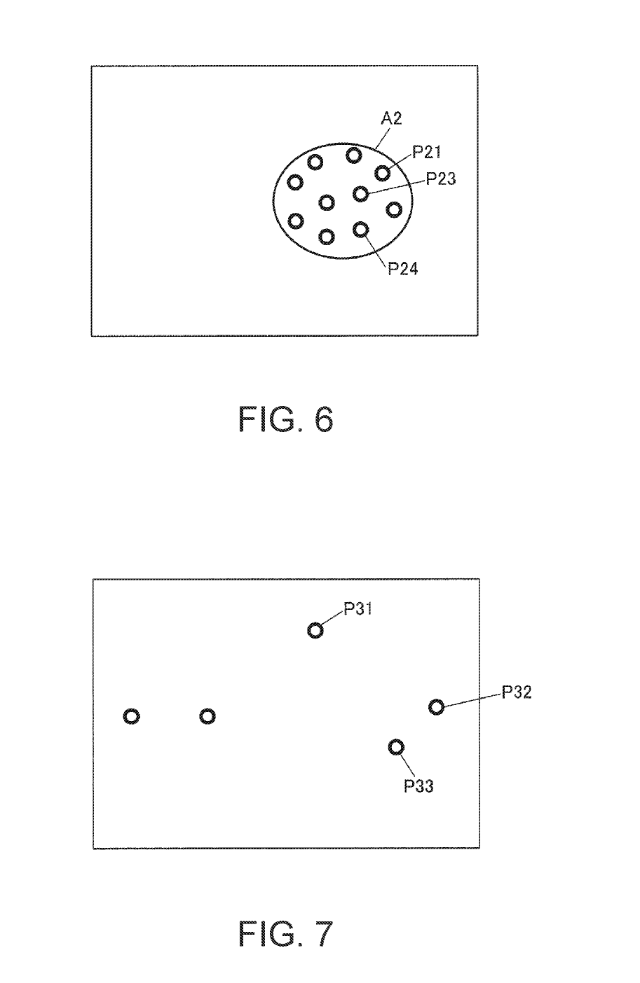

The number of desired sensitivity attenuation positions is not limited to one. Alternatively, multiple desired sensitivity attenuation positions may be set. FIGS. 6 and 7 are views illustrating setting examples in which multiple desired sensitivity attenuation positions are set. FIG. 6 illustrates a mode in which multiple desired sensitivity attenuation positions P21, P22, P23, . . . are set in a strong reflecting region A2. A predetermined number of multiple desired sensitivity attenuation positions P21, P22, P23, . . . may be automatically set, or may be manually designated and set by a user.

In contrast, as illustrated in FIG. 7, multiple desired sensitivity attenuation positions P31, P32, P33, . . . may be discretely set in a generated ultrasonic image. Also, in this case, the desired sensitivity attenuation positions P31, P32, P33, . . . may be manually designated and set by a user. Alternatively, in a case where multiple strong reflecting regions are extracted from a provisional ultrasonic image, one or a predetermined number of desired sensitivity attenuation positions may be automatically set in each of the strong reflecting regions.

In a case where multiple desired sensitivity attenuation positions are set, an addition weight is calculated such that the null point NP faces the direction of each of the desired sensitivity attenuation positions. As illustrated in FIG. 3, multiple null points NP are present. In the modification example, an addition weight is calculated such that each desired sensitivity attenuation position faces a separate null point NP. Specifically, when calculating an addition weight according to Expression (8), the weight calculation unit 243 applies constrained conditions represented by Expressions (12), (13), and (14) in which the direction of an undesired wave is set at θₖ, instead of the constrained conditions represented by Expressions (9), (10), and (11). k represents the number of desired sensitivity attenuation positions.

$$\begin{matrix}a = [ \begin{matrix}{a}_{0} & {a}_{θ 1} & {a}_{θ 2} & … & {a}_{θ k}\end{matrix} ] & ( 12 ) \\ {a}_{θ k} = ( \begin{matrix}1 & {e}^{- jπ si n ( θ k )} & {e}^{- j 2 π si n ( θ k )} & … & {e}^{- j ( M - 1 ) π si n ( θ k )}\end{matrix} ) & ( 13 ) \\ H = [ \begin{matrix}1 \\ {H}_{1} \\ {H}_{2} \\ ⋮ \\ {H}_{k}\end{matrix} ] \\ where {H}_{1} = 1 , {H}_{i} = 0 ( 1 < i ≦ k ) & ( 14 )\end{matrix}$$

According to the modification example, when a beam forming process is performed for a corresponding spot of each desired sensitivity attenuation position on a processing target line, it is possible to calculate an addition weight for the null points NP to respectively face the directions of the desired sensitivity attenuation positions, and to add a received signal of each element in a weighted manner using the calculated addition weight. Accordingly, it is possible to perform the beam forming process while reliably attenuating the signal strength of the received signal corresponding to each desired sensitivity attenuation position, and to obtain the same effects as those in the embodiment. As illustrated in FIG. 6, in a case where multiple desired sensitivity attenuation positions are set in a strong reflecting region inside a generated ultrasonic image, it is possible to concentrate the null points NP in the strong reflecting region, and thus, it is possible to effectively reduce the effects of reflected waves from the corresponding reflecting object, and to prevent the occurrence of a virtual image.

In the embodiment, adaptive beam forming is exemplified as a technique by which the null point NP faces the direction of a desired sensitivity attenuation position; however, the invention is not limited to the adaptive beam forming. For example, it is possible to calculate an addition weight using Schelkunoff array polynomial for the null point NP to face the direction of a desired sensitivity attenuation position.

In the embodiment, ultrasonic measurement is performed via a linear scanning method. Alternatively, the invention can be also applied similarly in a case in which scanning is performed via other methods such as sector scanning and offset sector scanning.

The entire disclosure of Japanese Patent Application No. 2015-230321 filed on Nov. 26, 2015 is expressly incorporated by reference herein.

What is claimed is:

1. An ultrasonic imaging device that generates an ultrasonic image by performing abeam forming process in which received ultrasonic signals are added, the device comprising:
a position setting unit that sets a desired sensitivity attenuation position in a generated ultrasonic image; and
a computational processing unit that executes the beam forming process while performing attenuation control to attenuate the signal strength of a received ultrasonic signal corresponding to the desired sensitivity attenuation position.

2. The ultrasonic imaging device according to claim 1,
wherein the beam forming process is performed for each scanning line of ultrasonic waves, and
wherein the computational processing unit specifies a corresponding spot of the desired sensitivity attenuation position on a processing target scanning line, and performs the attenuation control when the beam forming process is performed for the corresponding spot.

3. The ultrasonic imaging device according to claim 2,
wherein the computational processing unit detects a distance between a base point of the processing target scanning line and the desired sensitivity attenuation position, and specifies the corresponding spot on the processing target scanning line based on the distance.

4. The ultrasonic imaging device according to claim 2,
wherein the computational processing unit detects the direction of the desired sensitivity attenuation position with respect to the processing target scanning line, calculates an addition weight such that a null point of the receive sensitivity of the received ultrasonic signal faces the direction of the desired sensitivity attenuation position, and adds received ultrasonic signals in a weighted manner using the calculated addition weight.

5. The ultrasonic imaging device according to claim 1,
wherein the position setting unit sets multiple desired sensitivity attenuation positions in a predetermined region inside the generated ultrasonic image.

6. The ultrasonic imaging device according to claim 1, further comprising:
a provisional image generation unit that generates a provisional ultrasonic image based on the received ultrasonic signals,
wherein the position setting unit extracts a strong reflecting region from the provisional ultrasonic image, and sets the desired sensitivity attenuation position in the strong reflecting region.

7. The ultrasonic imaging device according to claim 1, further comprising:
a provisional image generation unit that generates a provisional ultrasonic image based on the received ultrasonic signals,
wherein the position setting unit receives an operation input indicating a position in the provisional ultrasonic image, and sets the desired sensitivity attenuation position.

8. A method of generating an ultrasonic image in which an ultrasonic image is generated by performing a beam forming process in which received ultrasonic signals are added, the method comprising:
setting a desired sensitivity attenuation position in a generated ultrasonic image; and
executing the beam forming process while performing attenuation control to attenuate the signal strength of a received ultrasonic signal corresponding to the desired sensitivity attenuation position.
